(19) United States
(12) Patent Application Publication
(10) Pub. No.: US 2024/0415929 A1
(43) Pub. Date: Dec. 19, 2024

(54) FORMULATIONS COMPRISING A TRIS BUFFER AND A PROTEIN

(71) Applicant: Amgen Inc., Thousand Oaks, CA (US)

(72) Inventors: Yael WEXLER-COHEN, Thousand Oaks, CA (US); Robert Matthew FESINMEYER, Newbury Park, CA (US); Rahul Rajan KAUSHIK, Newbury Park, CA (US)

(73) Assignee: AMGEN INC., Thousand Oaks, CA (US)

(21) Appl. No.: 18/822,739

(22) Filed: Sep. 3, 2024

Related U.S. Application Data
(63) Continuation of application No. 17/288,139, filed on Apr. 23, 2021.
(60) Provisional application No. 62/751,333, filed on Oct. 26, 2018.

(51) Int. Cl. A61K 38/17; A61K 9/00; A61K 47/18; A61K 47/26; A61K 47/40

(52) U.S. Cl. CPC A61K 38/179; A61K 9/0048; A61K 47/18; A61K 47/26; A61K 47/40

(57) ABSTRACT

Protein formulations and methods of making and using such formulations are provided herein. The formulation can be an ophthalmic formulation, such as for intravitreal administration. In some embodiments, the formulation comprises a VEGFR-Fc fusion protein, such as aflibercept. In some embodiments, the formulation comprises a Tris buffer.

### RELATED APPLICATIONS

This application is a continuation of U.S. application Ser. No. 17/288,139, filed Apr. 23, 2021, which is a U.S. national stage filing under 35 U.S.C. § 371 of International Application No. PCT/US19/58144, filed Oct. 25, 2019, which claims priority to U.S. Provisional Application No. 62/751,333, filed on Oct. 26, 2018, all of which are hereby incorporated by reference in its entirety.

### SEQUENCE LISTING

The present application is being filed along with a Sequence Listing in electronic format. The Sequence Listing is provided as a file entitled A-2308-US03-CNT_SQL, created Sep. 3, 2024, which is 5 kb in size. The information in the electronic format of the Sequence Listing is incorporated herein by reference in its entirety.

### FIELD OF THE INVENTION

The instant disclosure relates to protein formulations and methods for making and using such formulations, such as a formulation with Tris as a buffering agent.

### BACKGROUND

Vascular endothelial growth factor (VEGF), also referred to as VEGF-A, is a signaling protein that promotes the growth of new blood vessels and binds to VEGFR-1 and VEGFR-2. VEGF has been shown to be upregulated in many tumors and has a role in angiogenesis. VEGF has also been shown to have a role in intraocular neovascularization, such as choroidal neovascularization (CNV), which is a significant aspect of wet age-related macular degeneration (AMD).

VEGF inhibitors, such as anti-VEGF antibodies and fragments and decoy receptors or chimeric receptors, have been developed as therapeutics for the treatment of various conditions, such as cancer and ocular disorders. For example, an anti-VEGF antibody and an anti-VEGF Fab are both commercially available as bevacizumab and ranibizumab, respectively. Also, commercially available is aflibercept, a VEGFR-Fc fusion protein or “VEGF-trap.”

Aflibercept is a fusion protein composed of an IgG1 Fc domain fused to the Ig domain 2 of VEGFR-1 and Ig domain 3 of VEGFR-2. Aflibercept is marketed as Eylea® (Regeneron, Tarrytown, NY) for the treatment of various ocular conditions, including wet type AMD, and is formulated for intravitreal administration. The fusion protein is also marketed as Zaltrap® (ziv-aflibercept) (Regeneron, Tarrytown, NY) for the treatment of certain types of cancer and is formulated for intravenous administration.

Ophthalmic formulations, and in particular intravitreal administration, can have additional safety concerns as compared to other administration routes, and thus, have more specific requirements. For example, impact to a subject due to inflammation or other adverse reactions can be severe, and thus, more specific requirements may be required. For example, a formulation for intravitreal administration may require a narrower range of permissible osmolarity. A formulation for intravitreal administration may require a lower threshold of permissible particulation, e.g., USP <789> versus USP <788>. It is also advantageous to have a formulation that provides increased stability.

The present disclosure provides formulations that meets the need for new protein formulations, e.g., VEGFR-Fc fusion formulations, or intravitreal formulations that are stable, have less aggregation, or have related advantages.

### SUMMARY

Provided herein are protein formulations and methods for making and using such formulations. In some embodiments, the formulation is suitable for intravitreal administration. In some embodiments, the protein is a VEGFR-Fc fusion protein.

In one embodiment, the formulation comprises a fusion protein comprising a domain of a vascular endothelial growth factor (VEGF) receptor and an Fc domain, a buffering agent, a stabilizer, and optionally, a surfactant and/or a tonicity agent. In one embodiment, the formulation comprises Tris as a buffering agent.

In some embodiments, the Tris concentration is from 0.1 mM to 50 mM, such as from 0.5 mM to 50 mM, from 1 mM to 50 mM, from 2.5 mM to 40 Mm, from 5 mM to 30 mM, or from 10 mM to 20 mM. In one embodiment, the Tris concentration is about 0.5 mM, about 1 mM, about 2.5 mM, about 5 mM, about 10 mM, about 20 mM, about 25 mM, about 30 mM, about 40 mM, or about 50 mM. The formulation can have a pH within the buffering capacity of Tris, such as a pH between 7.0 and 9.0.

In some embodiments, the formulation comprises a stabilizer that is an amino acid or sugar. In one embodiment, the formulation comprises two different stabilizers, such as two different sugars. In some embodiments, the formulation comprises sucrose and trehalose, or sucrose and a cyclodextrin, such as hydroxypropyl-β-cyclodextrin (HPBCD).

In some embodiments, the formulation comprises a surfactant, such as polysorbate 20, polysorbate 80, or Pluronic® F68. In some embodiments, the formulation does not comprise a surfactant.

In some embodiments, the formulation comprises a tonicity agent, such as sodium chloride or potassium chloride. In other embodiments, the formulation does not comprise a tonicity agent.

In some embodiments, the fusion protein is aflibercept. In some embodiments, the concentration of aflibercept is about 40 mg/ml.

### BRIEF DESCRIPTION OF THE DRAWINGS

FIG. 1 shows the SE-UHPLC main peak results for Formulations 1-3, representing the aggregation level of aflibercept, as described in Example 1.

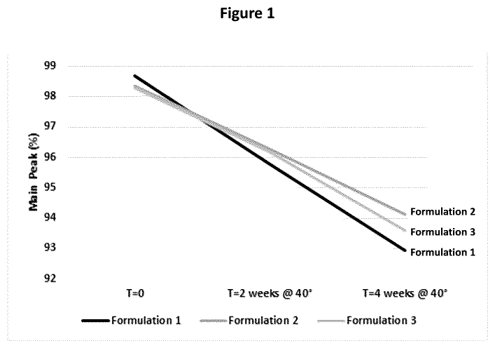

FIG. 2 shows the SE-UHPLC main peak results for Formulations 1, 4 and 5, representing the aggregation level of aflibercept, as described in Example 2.

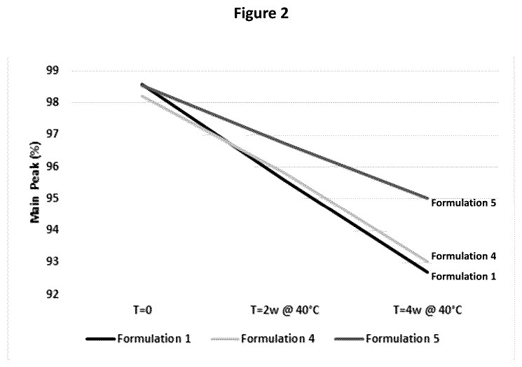

### DETAILED DESCRIPTION

The instant disclosure provides VEGFR-Fc fusion protein formulations and methods for making and using such formulations. In some embodiments, the formulation comprises a buffering agent, such as Tris.

In one embodiment, the formulation comprises a fusion protein comprising a domain of a vascular endothelial growth factor (VEGF) receptor and an Fc domain, Tris buffer, a stabilizer, and optionally, a surfactant and/or a tonicity agent. In one embodiment, the Tris buffer is from Tris HCl, Tris acetate, Tris citrate, Tris base, or Tris phosphate. In one embodiment, the concentration of the Tris or Tris buffer is from 0.1 mM to 50 mM, from 0.5 mM to 50 mM, between 1 mM to 50 mM, from 1 mM to 40 mM, from 2.5 mM to 40 mM, from 1 mM to 30 mM, from 1 mM to 20 mM, from 1 mM to 10 mM, from 1 mM to 5 mM, from 5 mM to 30 mM, or from 10 mM to 20 mM. In one embodiment, the concentration of the Tris or Tris buffer is about 0.5 mM, about 1 mM, about 2.5 mM, about 5 mM, about 10 mM, about 20 mM, about 25 mM, about 30 mM, about 40 mM, or about 50 mM.

In some embodiments, between 1 and 300 mg/ml of a fusion protein comprising a domain of a vascular endothelial growth factor (VEGF) receptor and an Fc domain is present in the formulations disclosed herein. In some embodiments, the formulations described herein comprises between 1 and 50 mg/ml, between 1 and 300 mg/ml, between 1 and 250 mg/ml, between 1 and 200 mg/ml, between 1 and 100 mg/ml of the fusion protein. In one embodiment, the formulation comprises between 10 and 50 mg/ml of the fusion protein. In one embodiment, the formulation comprises less than 300 mg/ml, less than 250 mg/ml, less than 200 mg/ml, less than 100 mg/ml, less than 50 mg/ml, less than 45 mg/ml, less than 40 mg/ml, less than 30 mg/ml, or less than 25 mg/ml of the fusion protein. In one embodiment, the formulation comprises about 300 mg/ml, about 250 mg/ml, about 200 mg/ml, about 100 mg/ml, about 50 mg/ml, about 45 mg m/, about 40 mg/ml, about 30 mg/ml, or about 25 mg/ml of the fusion protein. In one embodiment, the formulation comprises about 40 mg/ml of the fusion protein.

In some embodiments, the fusion protein comprises a domain of VEGFR1, a domain of VEGFR2, or a combination thereof. In some embodiments, the fusion protein comprises a domain of VEGFR1 and a domain of VEGFR2. In one embodiment, the fusion protein comprises Ig domain 2 of VEGFR1 and Ig domain 3 of VEGFR2. In one embodiment, the fusion protein comprises Ig domain 2 of VEGFR1, Ig domain 3 of VEGFR2, and an Fc domain of IgG1. In one embodiment, the fusion protein is a VEGF Trap. In another embodiment, the fusion protein is aflibercept. In another embodiment, the fusion protein comprises an amino acid sequence of SEQ ID NO: 1. In another embodiment, the fusion protein comprises an amino acid sequence of SEQ ID NO: 2. In one embodiment, the formulation comprises about 40 mg/ml of aflibercept. In one embodiment, the formulation comprises about 40 mg/ml of a fusion protein comprising a protein having an amino acid sequence of SEQ ID NO: 1 or SEQ ID NO: 2. In one embodiment, the formulation comprises about 40 mg/ml of a fusion protein comprising a protein having an amino acid sequence of SEQ ID NO: 1 and a fusion protein comprising a protein having an amino acid sequence of SEQ ID NO: 2.

SEQ ID NO: 1
SDTGRPFVEMYSEIPEIIHMTEGRELVIPCRVTSPNITVTLKKFPLDTLI
PDGKRIIWDSRKGFIISNATYKEIGLLTCEATVNGHLYKTNYLTHRQTNT
IIDVVLSPSHGIELSVGEKLVLNCTARTELNVGIDFNWEYPSSKHQHKKL
VNRDLKTQSGSEMKKFLSTLTIDGVTRSDQGLYTCAASSGLMTKKNSTFV
RVHEKDKTHTCPPCPAPELLGGPSVFLFPPKPKDTLMISRTPEVTCVVVD
VSHEDPEVKFNWYVDGVEVHNAKTKPREEQYNSTYRVVSVLTVLHQDWLN
GKEYKCKVSNKALPAPIEKTISKAKGQPREPQVYTLPPSRDELTKNQVSL
TCLVKGFYPSDIAVEWESNGQPENNYKTTPPVLDSDGSFFLYSKLTVDKS
RWQQGNVFSCSVMHEALHNHYTQKSLSLSPG
SEQ ID NO: 2
(SEQ ID NO: 2)
SDTGRPFVEMYSEIPEIIHMTEGRELVIPCRVTSPNITVTLKKFPLDTLI
PDGKRIIWDSRKGFIISNATYKEIGLLTCEATVNGHLYKTNYLTHRQTNT
IIDVVLSPSHGIELSVGEKLVLNCTARTELNVGIDFNWEYPSSKHQHKKL
VNRDLKTQSGSEMKKFLSTLTIDGVTRSDQGLYTCAASSGLMTKKNSTFV
RVHEKDKTHTCPPCPAPELLGGPSVFLFPPKPKDTLMISRTPEVTCVVVD
VSHEDPEVKFNWYVDGVEVHNAKTKPREEQYNSTYRVVSVLTVLHQDWLN
GKEYKCKVSNKALPAPIEKTISKAKGQPREPQVYTLPPSRDELTKNQVSL
TCLVKGFYPSDIAVEWESNGQPENNYKTTPPVLDSDGSFFLYSKLTVDKS
RWQQGNVFSCSVMHEALHNHYTQKSLSLSPGK

In one embodiment, the formulation comprises a fusion protein comprising a domain of a vascular endothelial growth factor (VEGF) receptor and an Fc domain (e.g., aflibercept, such as about 40 mg/ml of aflibercept), a buffering agent (e.g., Tris, such as between 2.5 mM to 50 mM of Tris, such as 10 mM), and optionally, a stabilizer and/or a surfactant.

In some embodiments, the formulation has a pH that is between 6.0 and 10.0. In some embodiments, the formulation has a pH between 6.0 and 9.5 or between 7.0 and 9.0. In some embodiments, the formatulion has a pH that is about 6.0, about 7.0, about 7.1, about 7.2, about 7.3, about 7.4, about 7.5, about 7.6, about 7.7, about 7.8, about 7.9, about 8.0, about 8.1, about 8.2, about 8.3, about 8.4, about 8.5, about 8.6, about 8.7, about 8.8, about 9.0, about 9.1, about 9.2, about 9.3, about 9.4, about 9.5, about 9.6, about 9.7, about 9.8, about 9.9, or about 10.0. In some embodiments, the pH is 7.0±0.3, 7.1±0.3, 7.2±0.3, 7.3±0.3, 7.4±0.3, 7.5±0.3, 7.6±0.3, 7.7±0.3, 7.8±0.3, 7.9±0.3, 8.0±0.3, 8.1±0.3, 8.2±0.3, 8.3±0.3, 8.4±0.3, 8.5±0.3, 8.6±0.3, 8.7±0.3, 8.8±0.3, 8.9±0.3, 9.0±0.3, 9.1±0.3, 9.2±0.3, 9.3±0.3, 9.4±0.3, 9.5±0.3, 9.6±0.3, 9.7±0.3, 9.8±0.3, 9.9±0.3, or 10.0±0.3.

In some embodiments, the formulation with Tris as a buffering agent is able to maintain a stable pH. For example, the formulation has a pH that is within about 0.1 or about 0.2 pH units after storage at one or two weeks at about 40° C. In some embodiments, the VEGFR-Fc fusion protein has increased stability as compared to a corresponding formulation with a different buffering agent (e.g., not Tris). The stability of the VEGFR-Fc fusion protein may be demonstrated by reduced aggregation levels, such as by Size Exclusion Ultra High Performance Liquid Chromatography (SE-UHPLC). The VEGFR-Fc fusion protein can be aflibercept. In some embodiments, the concentration of the protein is about 40 mg/mL. In one embodiment, the formulation also comprises a stabilizer, and optionally, a surfactant and/or a tonicity agent.

In one embodiment, the stabilizer is an amino acid. In one embodiment, the amino acid is proline. In another embodiment, the amino acid is glycine. In some embodiments, the amino acid is a basic amino acid, such as arginine or lysine. In other embodiments, the amino acid is an acidic amino acid, such as aspartic acid. In yet other embodiments, the amino acid is a hydrophobic amino acid, such as alanine. In some embodiments, the formulation comprises two different amino acids. In one embodiment, the stabilizer is a sugar. The sugar can be sucrose, sorbitol, glycerol, trehalose (e.g., α, α-trehalose or trehalose dihydrate), mannitol, dextrose, dextran, glucose, or any combination thereof. In one embodiment, the stabilizer is sucrose. In another embodiment, the stabilizer is trehalose. In another embodiment, the stabilizer is a cyclodextrin, such as hydroxypropyl-β-cyclodextrin (HPBCD). In yet another embodiment, the formulation comprises two different sugars, such as sucrose and trehalose, or sucrose and a cyclodextrin, such as HPBCD. In yet another embodiment, the formulation comprises one or more sugars and one or more amino acids.

The concentration of the stabilizer can be between 1 mM to 300 mM, between 10 mM to 300 mM, between 100 mM to 300 mM, between 200 mM to 300 mM, and between 200 mM and 280 mM. In one embodiment, the concentration of the stabilizer is about 200 mM, such as about 200 mM proline. In another embodiment, the concentration of the stabilizer is about 280 mM, such as about 280 mM glycine.

In yet other embodiments, the formulation comprises between 0 and 50% (w/v) of the stabilizer. In some embodiments, the formulation comprises between 0 and 25% (w/v) of the stabilizer. In some embodiments, the formulation comprises between 0 and 20% (w/v), between 5 and 50% (w/v), between 10 and 20% (w/v), between 0 and 10% (w/v), between 5 and 10% (w/v) or between 2 and 10% (w/v) of a stabilizer. In some embodiments, the formulation comprises about 1.5%, about 2%, about 2.5%, about 3%, about 3.5%, about 4%, about 4.5%, about 5%, about 5.5%, about 6%, about 6.5%, about 7%, about 7.5%, about 8%, about 8.5%, about 9%, about 9.5%, about 10%, about 11%, about 12%, about 13%, about 14%, about 15%, about 16%, about 17%, about 18%, about 19%, or about 20% (w/v) of a stabilizer, such as a sugar. The sugar can be sucrose, trehalose or a cyclodextrin, such as HPBCD. In one embodiment, the formulation comprises about 1.5%, about 2%, about 2.5%, about 3%, about 3.5%, about 4%, about 4.5%, about 5%, about 5.5%, about 6%, about 6.5%, about 7%, about 7.5%, about 8%, about 8.5%, about 9%, about 9.5%, or about 10% (w/v) of sucrose. In one embodiment, the formulation comprises about 5% sucrose. In yet another embodiment, the formulation comprises about about 1.5%, about 2%, about 2.5%, about 3%, about 3.5%, about 4%, about 4.5%, about 5%, about 5.5%, about 6%, about 6.5%, about 7%, about 7.5%, about 8%, about 8.5%, about 9%, about 9.5%, or about 10% (w/v) of trehalose. In one embodiment, the formulation comprises about 3% (w/v) trehalose. In one embodiment, the formulation comprises about 3.5% (w/v) trehalose. In one embodiment, the formulation comprises about 4% (w/v) trehalose. In one embodiment, the formulation comprises about 4.5% (w/v) trehalose. In one embodiment, the formulation comprises about 5% (w/v) trehalose. In another embodiment, the formulation comprises about 6.5% (w/v) trehalose.

In one embodiment, the formulation comprises two different sugars. In one embodiment, the concentration of the first sugar and the second sugar is each between 0 and 50% (w/v), between 0 and 25% (w/v), between 0 and 20% (w/v), between 5 and 50% (w/v), between 10 and 20% (w/v), between 0 and 10% (w/v), between 5 and 10% (w/v) or between 2 and 10% (w/v). In one embodiment, the total concentration of the first sugar and the second sugar is between between 0 and 50% (w/v), between 0 and 25% (w/v), between 0 and 20% (w/v), between 5 and 50% (w/v), between 10 and 20% (w/v), between 0 and 10% (w/v), between 5 and 10% (w/v) or between 2 and 10% (w/v). In another embodiment, the concentration of the first sugar is about about 1.5%, about 2%, about 2.5%, about 3%, about 3.5%, about 5%, about 6%, about 6.5%, about 7%, about 7.5%, about 8%, about 8.5%, about 9%, about 10%, about 11%, about 12%, about 13%, about 14%, about 15%, about 16%, about 17%, about 18%, about 19%, or about 20% (w/v). In another embodiment, the concentration of the second sugar is about 1.5%, about 2%, about 2.5%, about 3%, about 3.5%, about 5%, about 6%, about 6.5%, about 7%, about 7.5%, about 8%, about 8.5%, about 9%, about 10%, about 11%, about 12%, about 13%, about 14%, about 15%, about 16%, about 17%, about 18%, about 19%, or about 20% (w/v). In one embodiment, the first sugar is sucrose and the second sugar is trehalose.

In yet another embodiment, the formulation comprises about 1.5%, about 2%, about 2.5%, about 3%, about 3.5%, about 5%, about 6%, about 6.5%, about 7%, about 7.5%, about 8%, about 8.5%, about 9%, or about 10% (w/v) of sucrose and trehalose, such as about 1% (w/v) sucrose and about 7% (w/v) trehalose, about 2% (w/v) sucrose and about 6% (w/v) trehalose, about 3% (w/v) sucrose and about 5% (w/v) trehalose, about 4% (w/v) sucrose and about 4% (w/v) trehalose, about 5% (w/v) sucrose and about 3% (w/v) trehalose, about 6% (w/v) sucrose and about 2% (w/v) trehalose, or about 7% (w/v) sucrose and about 1% (w/v) trehalose. In another embodiment, the formulation comprises about 5% (w/v) sucrose and about 3.5% (w/v) trehalose, about 5% (w/v) sucrose and about 4% (w/v) trehalose, about 5% (w/v) sucrose and about 2.5% (w/v) trehalose, about 5% (w/v) sucrose and about 2% (w/v) trehalose, about 5% (w/v) sucrose and about 1.5% (w/v) trehalose, or about 4% (w/v) sucrose and about 2.5% (w/v) trehalose.

In yet another embodiment, the formulation comprises about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, or about 20% of sucrose and about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, or about 20% of a cyclodextrin, such as HPBCD, such as about 5% (w/v) sucrose and about 15% (w/v) of a cyclodextrin, such as HPBCD, about 5% (w/v) sucrose and about 16% (w/v) of a cyclodextrin, such as HPBCD, about 5% (w/v) sucrose and about 17% (w/v) of a cyclodextrin, such as HPBCD, about 5% (w/v) sucrose and about 18% (w/v) of a cyclodextrin, such as HPBCD, about 5% (w/v) sucrose and about 19% (w/v) of a cyclodextrin, such as HPBCD, or about 5% (w/v) sucrose and about 20% (w/v) of a cyclodextrin, such as HPBCD.

In one embodiment, the formulation does not comprise a surfactant. In another embodiment, the formulation comprises a fusion protein comprising a domain of a vascular endothelial growth factor (VEGF) receptor and an Fc domain (e.g., aflibercept, such as about 40 mg/ml of aflibercept), a buffering agent (e.g., Tris, such as between 2.5 mM to 40 mM of Tris, such as 10 mM), a stabilizer (e.g., sucrose and/or trehalose, or sucrose and a cyclodextrin, such as HPBCD), and a surfactant.

The surfactant can be a polyoxyethylene glycol alkyl ether, a polyoxypropylene glycol alkyl ether, a glucoside alkyl ether, a polyoxyethylene glycol octylphenol ether, a polyoxyethylene glycol alkylphenol ether, a glycerol alkyl ester, a polyoxyethylene glycol sorbitan alkyl ester, a sorbitan alkyl ester, a cocamide MEA, a cocamide DEA, a dodecyldimethylamine oxide, a poloxamer, a polyethoxylated tallow amine (POEA), or a combination thereof. In one embodiment, the surfactant is a polysorbate. In one embodiment, the surfactant is polysorbate 20. In another embodiment, the surfactant is polysorbate 80. In yet another embodiment, the surfactant is a poloxamer, such as poloxamer 188. In one embodiment, the surfactant is Pluronic® F-68. In some embodiments, the formulation comprises from 0.001 to 3% (w/v), 0.001 to 2% (w/v), 0.001 to 1% (w/v), 0.001 to 0.5% (w/v) or 0.01% to 0.1% (w/v) of a surfactant. In some embodiments, the formulation comprises about 0.01% (w/v) of a surfactant, such as polysorbate 80. In some embodiments, the formulation comprises about 0.005% (w/v) of a surfactant, such as polysorbate 80. In some embodiments, the formulation comprises about 0.03% (w/v) of a surfactant, such as polysorbate 20. In some embodiments, the formulation comprises about 0.1% (w/v) of a surfactant, such as Pluronic® F-68.

In one embodiment, the formulation comprises Tris (e.g., Tris HCl), sucrose, trehalose, and a surfactant and the pH is between 7.0 and 9.0, such as between 7.2 and 7.6 or between 7.3 and 7.5. In one embodiment, the formulation comprises about 10 mM Tris, about 5% (w/v) sucrose, about 3.5% (w/v) trehalose, and about 0.01% (w/v) polysorbate 80, at a pH of about 7.3. In one embodiment, the formulation comprises about 10 mM Tris, about 5% (w/v) sucrose, about 3.5% (w/v) trehalose, and about 0.01% (w/v) polysorbate 80, at a pH of about 7.5. In yet another embodiment, the formulation comprises about 10 mM Tris, about 5% (w/v) sucrose, about 18% (w/v) HPBCD, and about 0.01% (w/v) polysorbate 80, at a pH of about 7.3. In yet another embodiment, the formulation comprises about 10 mM Tris, about 5% (w/v) sucrose, about 18% (w/v) HPBCD, and about 0.01% (w/v) polysorbate 80, at a pH of about 7.5.

In one embodiment, the formulation does not comprise a tonicity agent. In another embodiment, the formulation comprises a fusion protein comprising a domain of a vascular endothelial growth factor (VEGF) receptor and an Fc domain (e.g., aflibercept, such as about 40 mg/ml of aflibercept), a buffering agent (e.g., Tris, such as between 2.5 mM to 40 mM of Tris, such as 10 mM), a stabilizer (e.g., sucrose and/or trehalose, or sucrose and a cyclodextrin, such as HPBCD), and a tonicity agent. In another embodiment, the formulation comprises a fusion protein comprising a domain of a vascular endothelial growth factor (VEGF) receptor and an Fc domain (e.g., aflibercept, such as about 40 mg/ml of aflibercept), a stabilizer (e.g., sucrose and/or trehalose, or sucrose and a cyclodextrin, such as HPBCD), a tonicity agent, and a surfactant (e.g., a polysorbate).

The concentration of the tonicity agent can be between 1 mM to 250 mM, between 5 mM to 200 mM, between 40 mM to 200 mM, or between 40 mM to 150 mM. In one embodiment, the concentration of the tonicity agent is about 1 mM, about 5 mM, about 10 mM, about 20 mM, about 30 mM, about 40 mM, about 50 mM, about 100 mM, about 140 mM or about 150 mM. The tonicity agent can be a salt, such as a chloride salt. In one embodiment, the tonicity agent is sodium chloride. In another embodiment, the tonicity agent is potassium chloride.

In some embodiments, a formulation disclosed herein may comprise an additional excipient. In one embodiment, the formulation can further comprise a polymeric excipient, such as hyaluronic acid, carboxymethylcellulose sodium (CMC), or poly (lactic-co-glycolic acid) (PLGA).

In some embodiments, the formulations disclosed herein are used for intravitreal administration, such as for the treatment of an ocular condition such as wet type age related macular degeneration (AMD). In some embodiments, the condition is macular edema following retinal vein occlusion (RVO) or diabetic retinopathy (DR). In yet other embodiments, the condition cause blindness. In one embodiment, the formulation is capable to be used with a prefilled syringe. In one embodiment, the prefilled syringe is for intravitreal administration of the formulation.

In some embodiments, the formulation disclosed herein has a particulate count (e.g., subvisible particle level or count) of less than 100 particles, less than 75 particles, less than 50 particles, less than 25 particles, less than 20 particles, less than 15 particles, less than 10 particles, less than 5 particles, or less than 2 particles, per one milliliter, for a particle size of ≥10 μm. In some embodiments, the formulation disclosed herein has a particulate count of less than 100 particles, less than 75 particles, less than 50 particles, less than 25 particles, less than 20 particles, less than 15 particles, less than 10 particles, less than 5 particles, or less than 2 particles, per one milliliter for a particle size of ≥25 μm. In some embodiments, the formulation disclosed herein has a particulate count of less than 100 particles, less than 75 particles, less than 50 particles, less than 25 particles, less than 20 particles, less than 15 particles, less than 10 particles, less than 5 particles, or less than 2 particles, per one milliliter, for a particle size of ≥50 μm. In some embodiments, the formulation disclosed herein has a particulate count of less than 50 particles per one milliliter for particle size of ≥10 μm. In some embodiments, the formulation disclosed herein has a particulate count of less than 5 particles per one milliliter for particle size of ≥25 μm. In some embodiments, the formulation disclosed herein has a particulate count of less than 2 particles per one milliliter for particle size of ≥50 μm. In some embodiments, the formulation disclosed herein has a particulate count of no more than an average of 50 particles per one milliliter for particle size of ≥10 μm. In some embodiments, the formulation disclosed herein has a particulate count of no more than an average of 5 particles per one milliliter for particle size of ≥25 μm. In some embodiments, the formulation disclosed herein has a particulate count of no more than an average of 2 particles per one milliliter for particle size of ≥50 μm.

In some embodiments, the particulate count is determined by light obscuration, such as through the use of a liquid particle counter, such as a commercially available counter developed by HIAC. In some embodiments, the particulate count is determined at a temperature of 25° C. In some embodiments, a first formulation (e.g., a Tris buffer formulation) is determined to be more desirable than a second formulation (e.g., a formulation without a Tris buffer) when there are fewer particulate counts or lower subvisible particle counts in the first formulation as compared to the second formulation. In another embodiment, a first formulation has similar particulate counts or subvisible particle counts (e.g., a lack of significance difference) as the second formulation.

In some embodiments, a first formulation (e.g., a Tris buffer formulation) is determined to be more stable than a second formulation (e.g., a formulation without a Tris buffer) when the fusion protein of the first formulation retains more of its original characteristics or properties than the fusion protein of the second formulation after one or more process stresses and/or after storage for a given time period. Stability of a formulation can be determined by analyzing the properties or characteristics of the protein such as known in the art, for example, as described in U.S. Pat. Nos. 8,092,803 and 9,982,032, and PCT Publications WO2017129685 and WO2018094316.

In one embodiment, a first formulation (e.g., a Tris buffer formulation) is determined to be more stable than a second formulation (e.g., a formulation without a Tris buffer) when the first formulation has less aggregation than the second formulation after one or more process stresses or stress conditions, such as known in the art, e.g., as described in WO2017129685. In one embodiment, the stress condition is shaking. In another embodiment, the stress condition is one or more freeze/thaw cycles, such as one, two, three, four or five freeze/thaw cycles. In another embodiment, the stress condition is vibration, pressure, and/or drop-shock. In one embodiment, the stress condition is photoexposure. In one embodiment, the stress condition is mixing. In one embodiment, the formulation is subjected to any one or more of the stress conditions. The stress conditions can comprise shaking, one or more freeze/thaw cycle(s), filtration, mixing, photoexposure, vibration, pressure, drop-shock stress, and/or any combination thereof. In one embodiment, the stress process comprises shaking (e.g., at 300 rpm at 25° C. for seven days); three freeze/thaw cycles from 25° C. to −20° C. with a rate of 1° C./min, and after each cooling/heating step the temperature is kept constant for ten minutes. In another embodiment, the stress process comprises three freeze/thaw cycles between 25° C. to −30° C.; filtration through a 0.2 μm PVDF filter; optionally, mixing; holding at 2° C. to 8° C., photoexposure, and a full transportation simulation (e.g., with a time sequence of more than 24 hours, 48 hours, 72 hours, 96 hours, or 110 hours, or between 24 and 110 hours, between 48 and 96 hours, such as about 50 hours, about 60 hours, about 70 hours, about 80 hours, about 90 hours, about 100 hours or about 100 hours, which includes vibration, pressure and drop-shock stresses).

In another embodiment, a first formulation (e.g., a Tris buffer formulation) is determined to be more stable than a second formulation (e.g., a formulation without a Tris buffer) when the first formulation has less aggregation than the second formulation after storage for about 1 week, about two weeks, about 3 weeks, about 4 weeks, about 3 months, about 4 months, about 5 months, about 6 months, about 7 months, about 8 months, about 9 months, about 10 months, about 11 months, about 12 months, about 15 months, about 18 months, about 21 months, about 24 months, about 30 months, or about 36 months. Storage can be at a given temperature, e.g., about 40° C., about 30° C., about 25° C., about 5° C., about −20° C. or about −30° C.

In one embodiment, a first formulation (e.g., a Tris buffer formulation) is more stable than a second formulation (e.g., a formulation without a Tris buffer) when the first formulation has less aggregation than the second formulation after one or more process stresses and storage for a given time period (e.g., about 1 week, about two weeks, about 3 weeks, about 4 weeks, about 3 months, about 4 months, about 5 months, about 6 months, about 7 months, about 8 months, about 9 months, about 10 months, about 11 months, about 15 months, about 18 months, about 21 months, about 24 months, about 30 months, or about 36 months, at about 40° C., about 30° C., about 25° C., about 5° C., about −20° C. or about −30° C.)

The stability of a formulation can be determined by any method known in the art, such as described in U.S. Pat. Nos. 8,092,803 and 9,982,032, and PCT Publications WO2017129685 and WO2018094316. In one embodiment, stability of a formulation is determined by chromatography, such as size exclusion chromatography, e.g., size exclusion high performance liquid chromatography (SE-HPLC) or size exclusion ultra high performance liquid chromatography (SE-UHPLC), or hydrophobic high performance liquid chromatography (HI-HPLC), in which a lower change or difference in a first peak from a first formulation before a stress process and/or storage condition as compared to a second peak from the same formulation after the stress process and/or storage condition as compared to a second formulation with a greater change or difference in its first and second peaks before and after a stress process and/or storage condition, respectively, indicates the first formulation is more stable than the second formation.

In another embodiment, stability of a formulation is determined by the turbidity of the formulation (e.g., such as measured at OD₄₀₅ nm), percent of protein recovered (e.g., determined by size exclusion HPLC (SE-HPLC)), and/or purity of protein (e.g., determined by SE-HPLC), in which lower turbidity, higher percentage of recovery and higher purity indicates higher stability. In some embodiments, SDS-PAGE (reducing or non-reducing) is used to determine the stability of a formulation. In some embodiments, asymmetric flow field-flow fractionation (AF4) is used. In other embodiments, isoelectric focusing (IEF), e.g., capiliary isoelectric focusing (cIEF), is used. Increased fragments and/or changes in IEF in a first formulation as compared to a second formulation would indicate the first formulation is less stable. Any one method or combination of methods can be used to determine the stability of a formulation.

The detailed description and following examples illustrate the present invention and are not to be construed as limiting the present invention thereto. Various changes and modifications can be made by those skilled in the art on the basis of the description of the invention, and such changes and modifications are also included in the present invention.

### EXAMPLES

### Example 1: Stability of Aflibercept in Tris Buffer Formulations

The stability of aflibercept in Tris formulations (Formulations 2 and 3 of Table 1 below) as compared to the Eylea® formulation (Formulation 1 of Table 1 below).

Forty mg/ml of aflibercept was buffer exchanged with the formulations specified in Table 1. Surfactant was added to the different formulations post the buffer exchange. Following the buffer exchange, the formulations underwent filtration, three freeze-thaw cycles (−30° C. and 25° C.) and drop-shock stresses. The performance of the buffer exchange was verified by testing osmolality, protein concentration and pH. The pH results following the buffer exchange demonstrated reduced pH value of 6.9 (“actual” pH in Table 1 below) versus the intended 7.3 for Formulation 2.

TABLE 1
Formulations 1-3.

| | Aflibercept | Buffering | Tonicity | | | |
|---|---|---|---|---|---|---|
| Formulation | (mg/mL) | Agent | Agent | Stabilizer | Surfactant | pH |
| 1 | 40 | 10 mM | 40 mM | 5% sucrose | 0.03% PS20 | 6.2 |
| | | sodium | NaCl | | | (actual |
| | | phosphate | | | | 6.3) |
| 2 | 40 | 10 mM Tris | | 5% sucrose, | 0.01% PS80 | 7.3 |
| | | HCl | | 3.5% trehalose | | (actual |
| | | | | dihydrate | | 6.9) |
| 3 | 40 | 10 mM Tris | | 5% sucrose, | 0.01% PS80 | 7.3 |
| | | HCl | | 18% HPBCD | | (actual |
| | | | | | | 7.2) |

To determine the protein stability of the formulations described in Table 1, the samples were stored at the stress condition of 40° C. for up to four weeks. The formulations were tested by Size Exclusion Ultra High Performance Liquid Chromatography (SE-UHPLC) to analyze the aggregation pattern post the buffer exchange and during storage. SE-UHPLC separates proteins based on differences in their hydrodynamic volumes. Molecules with larger hydrodynamic volumes elute earlier than molecules with smaller volumes. The samples were loaded onto an SE-UHPLC column, separated isocratically and the eluent monitored by UV absorbance. Purity was determined by calculating the percentage of each separated component as compared to the total integrated area. The higher the main peak value (e.g., represented as percentage of main peak) determined by SE-UHPLC for a formulation, the more stable the formulation, as it indicates a lower level of aggregation. Another indication of increased stability is a lack of change in the main peak value between an initial timepoint and a later timepoint as compared to another formulation.

The percentage of the main peak for each formulation in Table 1 was determined at timepoints 0, 2 weeks, and 4 weeks for the formulations stored at 40° C., as shown in Table 2. The difference or delta value between the main peak percentages of T=0 and T=2 weeks and between the main peak percentages of T=0 and T=4 weeks for the formulations is also shown in Table 2.

TABLE 2
SE-UHPLC Main Peak Results for Formulations 1-3.

| Formu- | | T = | Delta from | T = | Delta from |
|---|---|---|---|---|---|
| lation | T = 0 | 2 weeks | T = 0 | 4 weeks | T = 0 |
| 1 | 98.7 | 95.8 | 2.9 | 92.9 | 5.8 |
| 2 | 98.3 | 96.3 | 2.1 | 94.1 | 4.2 |
| 3 | 98.3 | 96.2 | 1.6 | 93.6 | 3.2 |

An improved stability profile is demonstrated by a reduction in aggregation levels, thus a higher main peak value and a lower delta value. Although the initial aggregation levels were slightly higher for the Tris formulations (i.e., Formulations 2 and 3 had slightly lower percentage of main peak values as compared to Formulation 1), surprisingly, the Tris formulations (Formulations 2 and 3) had reduced aggregation over storage at both two week and four weeks in comparison to Formulation 1, as demonstrated in Table 2. Both the main peak values at T=2 weeks and T=4 weeks was greater for the Tris formulations as compared to Formulation 1, and the change in main peak values for both Tris formulations from both 2 weeks and 4 weeks to T=0 were lower than that of Formulation 1.

In addition, the subvisible particle levels of the three formulations were tested by light obscuration (HIAC) to examine the impact of the formulation on the protein tendency to create particles. The results are shown in Table 3.

TABLE 3
Subvisible Particle Counts for Formulations 1-3.

| | 10 μm | | 25 μm | |
|---|---|---|---|---|
| | | T = 4w | | T = 4w |
| Formulation | T = 0 | (25° C.) | T = 0 | (25° C.) |
| 1 | 5 | 29 | 0 | 2 |
| 2 | 7 | 7 | 2 | 0 |
| 3 | 4 | 0 | 0 | 0 |

The Tris formulations (Formulations 2 and 3) exhibited reduced subvisible counts in major particle size

### Example 2: Stability of Aflibercept in Tris Buffer Formulation pH 7.5

As the pH result for Formulation 2 in Example 1 had a reduced pH value of 6.9 rather than the intended 7.3, another study was performed to test the Formulation 2 from Example 1 within the pH of buffering range of Tris (Formulation 4 of Table 4 below) in comparison with Formulation 1 from Example 2 (Formulation 1 of Table 4 below, which is the same as Formulation 1 of Table 1) as well as a formulation without any buffering agent (Formulation 5 of Table 4 below). The study was also performed on a larger scale. The formulations in Table 4 were prepared at a larger scale than those in Example 1 (formulations in Example 1 were buffer exchanged with a total volume of about 7 mL for each formulation, whereas formulations at larger scale were buffer exchanged with a total volume of about 60-100 mL for each formulation). Surfactant was added to the different formulations post the buffer exchange. Following the buffer exchange the formulations underwent filtration, three freeze-thaw cycles (−30° C. and 25° C.), photoexposure, and transportation simulation (including vibration pressure and drop-shock stresses).

The pH results following the buffer exchange at T=0 were 6.2, 7.6 and 6.3 for the three formulations, respectively (referred to as “actual” in Table 4).

TABLE 4
Formulations 1, 4 and 5.

| | Aflibercept | Buffering | Tonicity | | | |
|---|---|---|---|---|---|---|
| Formulation | (mg/mL) | Agent | Agent | Stabilizer | Surfactant | pH |
| 1 | 40 | 10 mM sodium | 40 mM | 5% sucrose | 0.03% | 6.2 (actual |
| | | phosphate | NaCl | | PS20 | 6.2) |
| 4 | 40 | 10 mM Tris | | 5% sucrose, 3.5% | 0.01% | 7.5 (actual |
| | | HCl | | trehalose dihydrate | PS80 | 7.6) |
| 5 | 40 | | | 5% sucrose, 3.5% | 0.01% | 6.2 (actual |
| | | | | trehalose dihydrate | PS80 | 6.3) |

To determine the protein stability in the formulations described in Table 4, the samples were stored at the stress condition of 40° C. for up to four weeks, storage condition of 25° C. for up to 24 weeks, storage condition of 5° C. for up to 52 weeks, and storage condition of −30° C. for up to 52 weeks. The formulations were tested by Size Exclusion Ultra High Performance Liquid Chromatography (SE-UHPLC) to analyze the aggregation pattern post the buffer exchange and during storage. SE-UHPLC separates proteins based on differences in their hydrodynamic volumes. Molecules with larger hydrodynamic volumes elute earlier than molecules with smaller volumes. The samples were loaded onto an SE-UHPLC column, separated isocratically and the eluent monitored by UV absorbance. Purity was determined by calculating the percentage of each separated component as compared to the total integrated area. The higher the main peak value (e.g., represented as percentage of main peak) determined by SE-UHPLC for a formulation, the more stable the formulation, as it indicates a lower level of aggregation. Another indication of increased stability is a lack of change in the main peak value between an initial timepoint and a later timepoint as compared to another formulation.

The percentage of the main peak for each formulation in Table 4 was determined at:
1. Timepoints 0, 1 week, 2 weeks, and 4 weeks for the formulations stored at 40° C., as shown in Table 5. The difference or delta value between the main peak percentages of T=0 and T=1 week, T=0 and T=2 weeks and T=0 and T=4 weeks for the formulations is also shown in Table 5.
2. Timepoints 2 weeks, 4 weeks, 12 weeks and 24 weeks for the formulations stored at 25° C., as shown in Table 6. The difference or delta value between the main peak percentages of T=0 and T=2 weeks, T=0 and T=4 weeks, T=0 and T=12 weeks and T=0 and T=24 weeks for the formulations is also shown in Table 6.
3. Timepoints 4 weeks, 12 weeks, 24 weeks and 52 weeks for the formulations stored at 5° C., as shown in Table 7. The difference or delta value between the main peak percentages of T=0 and T=4 weeks, T=0 and T=12 weeks, T=0 and T=24 weeks, and T=0 and T=52 weeks for the formulations is also shown in Table 7.
4. Timepoints 12 weeks, 24 weeks and 52 weeks for the formulations stored at −30° C., as shown in Table 8. The difference or delta value between the main peak percentages of T=0 and T=12 weeks, T=0 and T=24 weeks, and T=0 and T=52 weeks for the formulations is also shown in Table 8.

TABLE 5
SE-UHPLC Main Peak Results for the Formulations in Table 4 at 40° C.

| | | | Delta | | Delta | | Delta |
|---|---|---|---|---|---|---|---|
| | | T = 1 w | from | T = 2 w | from | T = 4 w | from |
| Formulation | T = 0 | (40° C.) | T = 0 | (40° C.) | T = 0 | (40° C.) | T = 0 |
| 1 | 98.6 | 97.2 | 1.4 | 95.6 | 3.0 | 92.7 | 5.9 |
| 4 | 98.2 | 97.1 | 1.1 | 95.8 | 2.4 | 93.0 | 5.2 |
| 5 | 98.5 | 97.7 | 0.9 | 96.7 | 1.8 | 95.0 | 3.5 |

TABLE 6
SE-UHPLC Main Peak Results for the Formulations in Table 4 at 25° C.

| | | | Delta | | Delta | | Delta | | Delta |
|---|---|---|---|---|---|---|---|---|---|
| | | T = 2 w | from | T = 4 w | from | T = 12 w | from | T = 24 w | from |
| Formulation | T = 0 | (25° C.) | T = 0 | (25° C.) | T = 0 | (25° C.) | T = 0 | (25° C.) | T = 0 |
| 1 | 98.6 | 98.5 | 0.1 | 98.3 | 0.3 | 98.0 | 0.6 | 97.8 | 0.7 |
| 4 | 98.2 | 97.9 | 0.3 | 97.9 | 0.4 | 97.4 | 0.8 | 97.0 | 1.2 |
| 5 | 98.5 | 98.4 | 0.2 | 98.2 | 0.3 | 97.8 | 0.8 | 97.7 | 0.9 |

TABLE 7
SE-UHPLC Main Peak Results for the Formulations in Table 4 at 5° C.

| | | | Delta | | Delta | | Delta | | Delta |
|---|---|---|---|---|---|---|---|---|---|
| | | T = 4 w | from | T = 12 w | from | T = 24 w | from | T = 52 w | from |
| Form. | T = 0 | (5° C.) | T = 0 | (5° C.) | T = 0 | (5° C.) | T = 0 | (5° C.) | T = 0 |
| 1 | 98.6 | 98.5 | 0.0 | 98.4 | 0.2 | 98.5 | 0.1 | 98.2 | 0.3 |
| 4 | 98.2 | 98.1 | 0.1 | 98.0 | 0.2 | 98.1 | 0.1 | 97.7 | 0.5 |
| 5 | 98.5 | 98.5 | 0.1 | 98.3 | 0.3 | 98.3 | 0.2 | 98.0 | 0.5 |

TABLE 8
SE-UHPLC Main Peak Results for the Formulations in Table 4 at −30° C.

| | | | Delta | | Delta | | Delta |
|---|---|---|---|---|---|---|---|
| | | T = 12 w | from | T = 24 w | from | T = 52 w | from |
| Form. | T = 0 | (−30° C.) | T = 0 | (−30° C.) | T = 0 | (−30° C.) | T = 0 |
| 1 | 98.6 | 98.5 | 0.1 | 98.7 | -0.1 | 98.5 | 0.0 |
| 4 | 98.2 | 98.1 | 0.2 | 98.3 | -0.1 | 98.2 | 0.0 |
| 5 | 98.5 | 98.4 | 0.2 | 98.6 | -0.1 | 98.4 | 0.1 |

In this study, the initial (T=0) aggregation level for the Tris formulation (Formulation 4) was slightly higher than Formulation 1 and the buffer free formulation, Formulation 5. The Tris formulation (Formulation 4) displayed a smaller increase in aggregation than Formulation 1 at the stress storage condition of 40° C. as demonstrated in Table 5. The change in main peak values for the Tris formulation (Formulation 4) from 1 week, 2 weeks and 4 weeks to T=0, respectively, were all lower than that of Formulation 1. The buffer-free formulation (Formulation 5) displayed the smallest increase in aggregation.

The increase in the aggregation level for the Tris formulation (Formulation 4) was similar to Formulation 1 (and Formulation 5) at the storage condition of 25° C., as demonstrated in Table 6. Despite having slight differences in the aggregation levels at this storage condition, all three formulations demonstrate an acceptable stability profile over the study duration of 52 weeks.

The aggregation level for the Tris formulation (Formulation 4) was comparable to Formulation 1 and the buffer free formulation, Formulation 5, at storage conditions of 5° C. and −30° C. as demonstrated in Table 7 and Table 8.

In summary all three formulations demonstrated acceptable aggregation stability profiles at all tested storage conditions while the Tris formulation (Formulation 4) demonstrated improved stability at the stress condition of 40° C. relative to Formulation 1.

In addition, the subvisible particle levels of the three formulations stored at 25° C. and 5° C. were tested by light obscuration (HIAC) to examine the impact of the formulation on the creation of particles. The results are shown in Tables 9 and 10, respectively.

TABLE 9
Subvisible Particle Counts for the Formulations in Table 4 at 25° C.

| | Time point and | Formu- | Formu- | Formu- |
|---|---|---|---|---|
| | Temperature | lation 1 | lation 4 | lation 5 |
| 10 μm | T = 0 | 2 | 12 | 14 |
| | T = 2 w @25° C. | 2 | 5 | 5 |
| | T = 4 w @25° C. | 0 | 5 | 0 |
| | T = 12 w @25° C. | 2 | 12 | 14 |
| | T = 24 w @25° C. | 48 | 52 | 63 |
| 25 μm | T = 0 | 0 | 4 | 0 |
| | T = 2 w @25° C. | 0 | 0 | 2 |
| | T = 4 w @25° C. | 0 | 0 | 0 |
| | T = 12 w @25° C. | 0 | 2 | 0 |
| | T = 24 w @25° C. | 12 | 22 | 7 |
| 50 μm | T = 0 | 0 | 4 | 0 |
| | T = 2 w @25° C. | 0 | 0 | 0 |
| | T = 4 w @25° C. | 0 | 0 | 0 |
| | T = 12 w @25° C. | 0 | 0 | 0 |
| | T = 24 w @25° C. | 2 | 5 | 2 |

TABLE 10
Subvisible Particle Counts for the Formulations in Table 4 at 5° C.

| | Time point and | Formu- | Formu- | Formu- |
|---|---|---|---|---|
| | Temperature | lation 1 | lation 4 | lation 5 |
| 10 μm | T = 4 w @5° C. | 2 | 0 | 9 |
| | T = 12 w @5° C. | 4 | 5 | 12 |
| | T = 24 w @5° C. | 50 | 42 | 17 |
| | T = 52 w @5° C. | 2 | 3 | 2 |
| 25 μm | T = 4 w @5° C. | 0 | 0 | 0 |
| | T = 12 w @5° C. | 0 | 0 | 0 |
| | T = 24 w @5° C. | 22 | 8 | 3 |
| | T = 52 w @5° C. | 0 | 0 | 0 |
| 50 μm | T = 4 w @5° C. | 0 | 0 | 0 |
| | T = 12 w @5° C. | 0 | 0 | 0 |
| | T = 24 w @5° C. | 3 | 3 | 0 |
| | T = 52 w @5° C. | 0 | 0 | 0 |

The Tris formulation (Formulation 4) exhibited higher subvisible counts in comparison to the Formulation 1, and similar subvisible counts in comparison to Formulation 5, for the 2 μm and 5 μm particle sizes in this study (data not shown). The 24 weeks results, for all three formulations for all particle sizes at both 5° C. and 25° C., are higher than the results for the other timepoints and the requirements specified in USP <789>. However, this may be a result of sample handling as the 52 weeks results are comparable to the other timepoints and are well within the USP <789> requirements. In summary, there was likely no significant difference in the counts for the 10 μm, 25 μm and 50 μm particle sizes, as can be seen in Tables 9 and 10, which indicates comparable stability profiles for all three formulation.

While the present invention has been described in terms of various embodiments, it is understood that variations and modifications will occur to those skilled in the art. Therefore, it is intended that the appended claims cover all such equivalent variations that come within the scope of the invention as claimed. In addition, the section headings used herein are for organizational purposes only and are not to be construed as limiting the subject matter described.

All references cited in this application are expressly incorporated by reference herein for any purpose.

1. A formulation comprising:
a) a fusion protein comprising a domain of a vascular endothelial growth factor (VEGF) receptor and an Fc domain;
b) a Tris buffer, wherein the Tris buffer concentration is between 1 mM and 50 mM; and
c) a stabilizer, wherein the stabilizer is an amino acid or sugar; and
wherein the formulation is suitable for intravitreal administration.

2. The formulation of claim 1, wherein the pH is between 6.0 and 10.0.

3. The formulation of claim 1, wherein the protein is aflibercept.

4. The formulation of claim 3, wherein the concentration of aflibercept is about 40 mg/ml.

5. The formulation of claim 1, wherein the stabilizer is proline, glycine, arginine, sucrose, cyclodextrin, or trehalose.

6. The formulation of claim 1, wherein the cyclodextrin is hydroxypropyl-β-cyclodextrin (HPBCD).

7. The formulation of claim 5, wherein the stabilizer is a sugar and the concentration of the sugar is between 2% and 10% (w/v) or between 5% and 50% (w/v).

8. The formulation of claim 7, further comprising a second sugar.

9. The formulation of claim 8, wherein the concentration of the second sugar is between 2% and 10% (w/v).

10. The formulation of claim 9, wherein the first sugar is sucrose and second sugar is trehalose.

11. The formulation of claim 10, wherein the concentration of sucrose is about 5% (w/v) and the concentration of trehalose is about 3.5% (w/v).

12. The formulation of claim 9, wherein the first sugar is sucrose and the second sugar is a cyclodextrin.

13. The formulation of claim 12, wherein the concentration of sucrose is about 5% (w/v) and the concentration of the cyclodextrin is about 18% (w/v).

14. The formulation of claim 13, wherein the cyclodextrin is HPBCD.

15. The formulation of any claim 1, further comprising a surfactant.

16. The formulation of claim 15, wherein the surfactant is polysorbate 20, polysorbate 80 or Pluronic® F68.

17. The formulation of claim 16, wherein the concentration of the surfactant is between 0.001% and 0.1%.

18. The formulation of claim 17, wherein the concentration of the surfactant is about 0.01% and the surfactant is polysorbate 80.

19. The formulation of claim 1, further comprising a tonicity agent.

20. The formulation of claim 19, wherein the tonicity agent is sodium chloride or potassium chloride and the concentration of the tonicity agent is between 1 mM and 150 mM.


SEQUENCE LISTING (2 sequences)

SEQ ID NO: 1 - protein, 431 aa, synthetic construct
  REGION 1..431: note = Fusion protein
SDTGRPFVEMYSEIPEIIHMTEGRELVIPCRVTSPNITVTLKKFPLDTLIPDGKRIIWDSRKGFIISNATYKEIGLLTCEATVNGHLYKTNYLTHRQTNTIIDVVLSPSHGIELSVGEKLVLNCTARTELNVGIDFNWEYPSSKHQHKKLVNRDLKTQSGSEMKKFLSTLTIDGVTRSDQGLYTCAASSGLMTKKNSTFVRVHEKDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 2 - protein, 432 aa, synthetic construct
  REGION 1..432: note = Fusion protein
SDTGRPFVEMYSEIPEIIHMTEGRELVIPCRVTSPNITVTLKKFPLDTLIPDGKRIIWDSRKGFIISNATYKEIGLLTCEATVNGHLYKTNYLTHRQTNTIIDVVLSPSHGIELSVGEKLVLNCTARTELNVGIDFNWEYPSSKHQHKKLVNRDLKTQSGSEMKKFLSTLTIDGVTRSDQGLYTCAASSGLMTKKNSTFVRVHEKDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK